(19) United States
(12) Patent Application Publication
(10) Pub. No.: US 2013/0071395 A1
(43) Pub. Date: Mar. 21, 2013

(54) COMPOSITIONS AND METHODS FOR TREATING PATHOLOGIES

(71) Applicant: James Basilion, Shaker Heights, OH (US)

(72) Inventor: James Basilion, Shaker Heights, OH (US)

(73) Assignee: CASE WESTERN RESERVE UNIVERSITY, Cleveland, OH (US)

(21) Appl. No.: 13/700,588

(22) PCT Filed: May 31, 2011
(86) PCT No.: PCT/US11/38551

§ 371 Date: Nov. 28, 2012

Related U.S. Application Data
(60) Provisional application No. 61/349,435, filed on May 28, 2010.

(51) Int. Cl. C07K 16/46

(52) U.S. Cl. CPC C07K 16/46
USPC 424/134.1; 530/387.3

(57) ABSTRACT

A composition for treating a neoplastic disorder includes an activatable cell penetrating peptide coupled to antibody and/or fragment thereof to an essential gene product of a neoplastic cell.

### RELATED APPLICATION

This application claims priority from U.S. Provisional Application No. 61/349,435, filed May 28, 2010, the subject matter of which are incorporated herein by reference in their entirety.

### TECHNICAL FIELD

This application generally relates to compositions and methods for treating pathologies, such as neoplastic disorders, and more particularly to compositions including a neutralizing antibody and/or fragment thereof to an essential cellular gene product and an activatable cell penetrating peptides capable of selectively transporting the neutralizing antibodies and/or fragments thereof across neoplastic cell membranes.

### BACKGROUND

Selectivity of therapeutic cancer agents is a desirable feature for limiting the adverse side effects from unrestricted exposure to a therapeutic agent and for enhancing the effectiveness of treatment. In addition to designing therapeutic agents with high specificity for the intended molecular target, selectivity may also be achieved by controlled transport through biological barriers and selective activation of the therapeutic agent.

Controlling transport of the therapeutic agent through biological barriers provides one basis for selectively delivering the therapeutic agent to the intended target. Strategies for selective transport include use of a targeting component that directs the agent to a specific cell surface molecule, which is then internalized via regulated cellular transport mechanisms. One method includes use of antibodies selective for a unique cell surface antigen or use of ligands selective for a receptor expressed on the surface of the targeted cell. This selective targeting approach, however, requires restricted presence of the cell surface marker on the cells being targeted for therapy. General expression of the cell surface antigen or receptor on non-targeted cells makes such targeted delivery less desirable while absence of specific markers on the cell surface severely limit this delivery strategy to only certain types of cancers.

Another strategy to enhance selectivity of a therapeutic agent is the use of an inactive compound (e.g., a prodrug), which is converted to the active form by chemical modification. In this approach, endogenous enzymes are exploited to convert the prodrug to the active compound. These strategies are effective if the prodrug or activated compound is itself capable of entering the targeted cell. Lack of permeability of the compounds can limit the use of this technique.

Over the last decade, peptide sequences that can readily enter a cell have been identified. For example, the Tat protein of the human immunodeficiency virus 1 (HIV-1) is able to enter cells from the extracellular environment. Such peptides, termed cell penetrating peptides (CPPs), are readily taken into cells and may be used to carry other molecules into cells. Peptides that are capable of facilitating transport of substances into cells have been termed cell penetrating peptides. The most important CPPs are rich in amino acids, such as arginine with positively-charged side chains. Molecules transported into cell by such cationic peptides may be termed “cargo” and may be reversibly or irreversibly linked to the cationic peptides. The uptake facilitated by CPPs is typically without specificity, enhancing uptake into most or all cells. Thus, although CPPs are capable of entering cells and may be capable of enhancing the transport of other molecules linked to CPPs into cells, control and regulation of such transport remains difficult.

### SUMMARY

This application relates to compositions and methods for treating pathologies, such as neoplastic disorders, and more particularly to composition that include antibodies and/or fragments thereof to essential gene products that are coupled to activatable cell penetrating peptides capable of selectively transporting antibodies and/or fragments thereof across cell membranes, such as neoplastic cell membranes.

According to one aspect of application, a composition can have the structure A-X—B-Ab. The Ab portion can be a neutralizing antibody and/or fragment thereof that binds to an essential gene product and/or neoplastic disorder-specific intracellular target. The B portion can be a polycationic domain comprising at least one basic amino acid residue, which is effective to transport the Ab portion across a membrane of at least one mammalian cell. The A portion can be a polyanionic domain comprising at least one acidic amino acid residue, which when linked with the B portion is effective to inhibit or prevent uptake of the B-Ab portions by at least one mammalian cell. The X portion can be a cleavable linker comprising a plurality of amino acid residues, which is cleavable by a gene expression product of at least one neoplastic cell.

Another aspect of the application relates to a method for treating a neoplastic disorder. The method can include administering to at least one neoplastic cell of the subject an effective amount of a composition that has the structure A-X—B-Ab. The Ab portion can be a neutralizing antibody and/or fragment thereof. The B portion can be a polycationic domain comprising at least one basic amino acid residue, which is effective to transport the Ab portion across a membrane of at least one mammalian cell. The A portion can be a polyanionic domain comprising at least one acidic amino acid residue, which when linked with the B portion is effective to inhibit or prevent uptake of the B-Ab portions by at least one mammalian cell. The X portion can be a cleavable linker comprising a plurality of amino acid residues, which is cleavable by a gene expression product of at least one neoplastic cell. The Ab portion can bind to an essential and/or neoplastic disorder-specific intracellular target and thereby prevent replication of the at least one neoplastic cell.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates schematic view of an activatable cell penetrating peptide comprising an acidic portion A, a basic portion B, a linker portion X, and an antibody and/or fragment thereof portion Ab according to one aspect of the application.

FIG. 2 illustrates a flow diagram showing a method for treating a neoplastic disorder according to another aspect of the application.

FIG. 3 illustrates a strategy for synthesis of Compound 1. The subsequent free amine is reacted with Cy5-NHS ester. Similar selective strategy was used for compound 3.

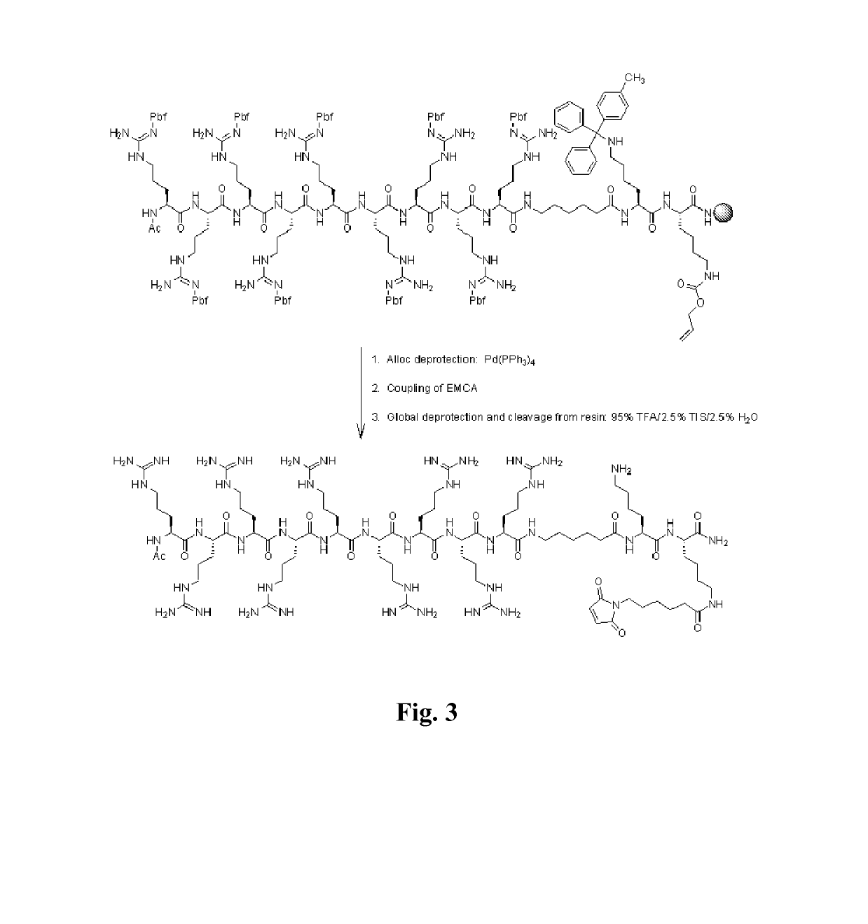

FIG. 4 illustrates structures of compounds 1, 2, and 3.

FIG. 5 illustrates a strategy for bioconjugation of cell penetrating antibody in accordance with an aspect of the application. Thiolane is incubated with pure immunoglobulin antibody (A). Thiolated antibody is incubated with compound 1 to generate an antibody with cell penetrating properties (B).

FIG. 6 illustrates images showing a time course study of cell penetrating peptide compound 1 in MDA MB-468 cells. Cell penetrating peptide with Cy5 are already in the cytosol after 4 hours of incubation. The nucleus is stained with DAPI. 20× objective

FIG. 7 illustrates images showing cell penetrating RPA1 antibody penetrates MDA MB 468 (top) and MCF-7 (bottom) cells. After 4 hours of incubation, the RPA1 antibody is in the cytosol. The nucleus is stained with DAPI and the mouse IgG RPA70 antibody is labeled with an anti-mouse AlexaFluor 488.

FIG. 8 illustrates images showing cell penetrating His6x antibody penetrates MDA MB 468 (top) and MCF-7 (bottom) cells. After 4 hours of incubation, the His6x antibody is in the cytosol. The nucleus is stained with DAPI and the mouse IgG His6x antibody is labeled with an anti-mouse IgG AlexaFluor 488.

FIG. 9 illustrates a chart showing cell penetrating mouse immunoglobulin antibody penetrates HT1080 cells. Compounds 1, 3, and non-specific mouse immunoglobulin antibody were incubated for 4 hours with MMP2 overexpressing HT1080 cells in 96-well plates. Cell viability was determined by cresyl violet assay. Treatment of cells did with the peptide compounds and cell penetrating non-specific mouse immunoglobulin antibody were not toxic after 4 hours of incubation.

### DETAILED DESCRIPTION

All scientific and technical terms used in this application have meanings commonly used in the art unless otherwise specified. The definitions provided herein are to facilitate understanding of certain terms used frequently herein and are not meant to limit the scope of the present invention.

As used herein, the term “neoplastic disorder” can refer to a disease state in a subject in which there are cells and/or tissues that proliferate abnormally. Neoplastic disorders can include, but are not limited to, cancers, sarcomas, tumors, leukemias, lymphomas, and the like.

As used herein, the term “neoplastic cell” can refer to a cell that shows aberrant cell growth, such as increased, uncontrolled cell growth. A neoplastic cell can be a hyperplastic cell, a cell from a cell line that shows a lack of contact inhibition when grown in vitro, a tumor cell, or a cancer cell that is capable of metastasis in vivo. Alternatively, a neoplastic cell can be termed a “cancer cell.” Non-limiting examples of cancer cells can include melanoma, breast cancer, ovarian cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, thymoma, lymphoma cells, melanoma cells, sarcoma cells, leukemia cells, retinoblastoma cells, hepatoma cells, myeloma cells, glioma cells, mesothelioma cells, and carcinoma cells.

As used herein, the term “tumor” can refer to an abnormal mass or population of cells that result from excessive cell division, whether malignant or benign, and all pre-cancerous and cancerous cells and tissues.

As used herein, the terms “treating” or “treatment” of a disease (e.g., a neoplastic disorder) can refer to executing a treatment protocol to eradicate at least one neoplastic cell. Thus, “treating” or “treatment” does not require complete eradication of neoplastic cells.

As used herein, the term “subject” can refer to any animal, including, but not limited to, humans and non-human animals (e.g., rodents, arthropods, insects, fish), non-human primates, ovines, bovines, ruminants, lagomorphs, porcines, caprines, equines, canines, felines, ayes, etc.), which is to be the recipient of a particular therapeutic application.

As used herein, the term “polypeptide” can refer to a molecule composed of monomers (amino acids) linearly linked by amide bonds (also known as peptide bonds). The term can indicate a molecular chain of amino acids and does not refer to a specific length of the product. Thus, peptides, dipeptides, tripeptides, oligopeptides, and proteins can be included within the definition of polypeptide. This term can also refer to the products of post-expression modifications of a polypeptide, such as glycosylation, hyperglycosylation, acetylation, phosphorylation, and the like. A polypeptide may be derived from a natural biological source or produced by recombinant technology, and is not necessarily translated from a designated nucleic acid sequence. A polypeptide may be generated by any manner known in the art, such as chemical synthesis.

As used herein, the term “translocation” can refer to transfer of at least a portion of an activated cell penetration peptide (ACPP) across a cell membrane such that at least a portion of the ACPP is internalized within the cell.

As used herein, the term “effective amount” can refer to the dosage of an ACPP, or a pharmaceutical composition including an ACPP, that is sufficient to provide treatment for a neoplastic disorder. The effective amount can vary depending on the subject, the neoplastic disorder being treated, and the treatment being affected.

This application relates to compositions and methods for treating pathologies, such as neoplastic disorders, and more particularly to compositions including antibodies and/or fragments thereof targeted to essential gene products that are coupled to ACPPs for targeting neoplastic cells or cancer cells. Many of the oldest and most efficacious cancer chemotherapeutic agents target cellular elements essential to the growth and proliferation of all cells (e.g., DNA with alkylating agents or dihydrofolate reductase with methotrexate), which underscores the utility of targeting essential biological molecules. Due to the shared necessity of these cellular targets for both normal and cancer cells, however, as well as the lack of drug targeting, toxicity of such drugs is high and results in side effects that limit their utility. Advantageously, the compositions described herein that include ACPPs can inactivate essential and/or neoplastic disorder-specific genes and/or gene products by selectively delivering neutralizing antibodies against the essential genes and/or gene products, such as intracellular genes and/or gene products, to perturb neoplastic cell growth and proliferation. Similar to chemotherapeutic agents, the composition of the present invention are only toxic to rapidly dividing cells. Unlike chemotherapeutic agents, however, the compositions of the present invention advantageously include a second level of specificity by only entering neoplastic cells that overexpress certain tumor associated gene products.

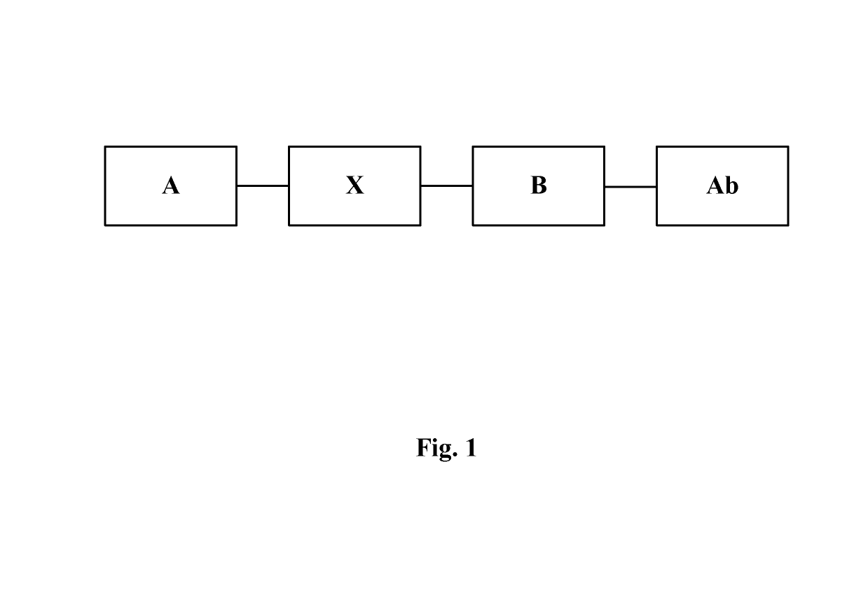

In one aspect of the application, the composition can include an antibody and/or fragment thereof that is coupled to an ACPP. In one example, the composition can have the structure A-X—B-Ab (FIG. 1). The A portion can include a polyanionic domain comprising at least one acid amino acid residue, the X portion can comprise a cleavable linker including a plurality of amino acid residues, the B portion can include a polycationic domain comprising at least one basic amino acid residue, and the Ab portion can comprise a neutralizing antibody and/or fragment thereof. As explained in more detail below, the intact structure is not significantly taken up by mammalian or non-neoplastic cells; however, upon extracellular cleavage of the X portion, the B-Ab portion can be taken up by a cell and thereby deliver the Ab portion to the cytosol and/or nucleus of the cell. Such controlled uptake and selective delivery of neutralizing antibodies and/or fragments thereof into cells may be useful, for example, in the treatment of neoplastic disorders.

Amino acids comprising the ACPP can include standard amino acids, non-standard amino acids, modified amino acids, and/or peptide mimetic moieties. Standard amino acids can include non-polar, aliphatic residue (e.g., glycine and alanine), aromatic residues (e.g., phenylalanine and tyrosine), polar, non-charged residues (e.g., serine and threonine), positively-charged residues (e.g., lysine and arginine), and negatively-charged residues (e.g., aspartate and glutamate). Examples of non-standard amino acids can include hydroxylysine, desmosine, and isodesmosine. Examples of modified amino acids can include post-translationally modified amino acids, such as methylated amino acids (e.g., methyl histidine, methylated forms of lysine, etc.), acetylated amino acids, amidated amino acids, formylated amino acids, hydroxylated amino acids, and phosphorylated amino acids. Examples of peptide mimetic moieties can include peptide portions linked by non-peptide bonds and amino acids linked by (or to) non-amino acid portions, such as peptoids, carbamates, vinyl polymers, or other molecules having non-peptide linkages but having an acidic portion cleavably linked to a basic portion.

In another aspect, the Ab portion can include a neutralizing antibody and/or fragment thereof that binds to an essential and/or neoplastic disorder-specific intracellular target. Neutralizing antibodies can include whole antibodies, e.g., of any isotype (IgG, IgA, IgM, IgE, etc) and/or fragments thereof which are also specifically reactive with an essential and/or neoplastic disorder-specific intracellular target. Neutralizing antibodies can be fragmented using conventional techniques and the fragments screened for utility and/or interaction with a specific epitope of interest. Thus, neutralizing antibodies and/or fragments thereof can include segments of proteolytically-cleaved or recombinantly-prepared portions of an antibody molecule and/or fragment thereof that is/are capable of reacting with an essential and/or neoplastic disorder-specific intracellular target. Non-limiting examples of such proteolytic and/or recombinant fragments can include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. Neutralizing antibodies and/or fragments thereof can include polyclonal, monoclonal, or other purified preparations of antibodies, recombinant antibodies, monovalent antibodies, and multivalent antibodies. Neutralizing antibodies and/or fragments thereof may be humanized and further include engineered complexes that comprise antibody-derived binding sites, such as diabodies and triabodies.

In one example, the Ab portion can comprise the neutralizing antibody directed to aSSB70C as disclosed by Basilion, J. P. et al, Mol Pharma. 56:359-369, 1999 (hereinafter, “Basilion et al.).

Essential intracellular targets can include genes and/or gene products that are needed for cell survival and do not have known redundant systems. Examples of essential intracellular targets can include those disclosed by Basilion et al., as well as those associated with the TCA cycle (e.g., the ACLY, ACO1, ACO2, CS, DLD, DLST, FH, IDH1, IDH2, IDH3A, IDH3B, MDH1, MDH2, OGDH, PC, PCK1, PCK2, SDHA, SDHB, SDHC, SDHD, SUCLA2, and SUCLG2 genes and associated gene products), iron metabolism (e.g., the HFE, RE1, FRE2, FET3, CTR1, FET4, CCC2, MAC1, IRP-1, IRP-2, MNK, and WD genes and associated gene products), heat shock proteins (e.g., the hsp70, hsp90, groE genes and their associated gene products), DNA replication (e.g., the cdc23, MCM10, Orc1, Cdc6, polD1, polD2, pri1, pri2, pcn, and rad2 genes and associated gene products), and ribosomal factors (e.g., the PRP43, rps29, rpl6, rps4, rpl35a, rps15a, rplp0, rps3, rpl38, rpl11, rpl35, rpl36a, rps3a, rplp2, rpl5, rps8, rpl28, rps19, rps15, rpl24, and rplp1 genes and associated gene products). Other examples of essential intracellular targets can include genes and/or gene products that are needed for cell survival and do not have known redundant systems are known in the art. (See for example, Dalgliesh, G L, et al., “Systematic sequencing of renal carcinoma reveals inactivation of histone modifying genes”, Nature. 2010; 463; 360-3; Dassain, M., et al., “A new essential gene of the minimal genome affecting cellular division” Biochemie. 81(8):889-895. 1999; Ayes, S J, et al., “The essential schizosaccharomyces pombe cdc23 DNA replication gene shares structural and functional homology with the Saccharomyces cerevisiae DNA43 (MCM10) gene” Current Genetics. 34(3):164-71. 1998; Berquist, B R, et al. “Essential and non-essential DNA replication genes in the model halophilic Archaeon, Halobacterium sp. NRC-1”, BMC Genetics. 8(31). 2007; Lindquist, S., et al., “The Heat-Shock Proteins”, Annual Review of Genetics. 22:631-77. 1988; Drakesmith, H. et al., “Viral infection and iron metabolism”, Nature Revies Microbiology. 6:541-552. 2008; De Silvia, D M, et al., “Molecular mechanisms of iron uptake in eukaryotes”, Physiological Reviews. 76:31-47. 1996; Combs, D J, et al, “Prp43p is a death-box splicesome disassembly factor essential for ribosome biogenesis”, Molecular and Cellular Biology. 26(2):523-534. 2006; Uechi, T, et al., “Ribosome gene protein knockdown causes developmental defects in zebrafish”, Plos One 1(1). 2006; Dassain, M., et al., “A new essential gene of the minimal genome affecting cellular division”, Biochemie. 81(8):889-895. 1999; Torres-Rosell, J., et al., “Can eukaryotic cells monitor the presence of unreplicated DNA”, Cell Division, 2:19. 2007, all of which are incorporated by reference in their entirety.)

In one example, an essential intracellular gene product can include the 70 kDa replication protein A1 (RPA70) as disclosed by Basilion et al.

Neoplastic disorder-specific intracellular targets can include genes and/or gene products that are specific to a particular neoplastic disorder. Examples of neoplastic disorder-specific genes and/or intracellular gene products are known in the art.

In another aspect of the present invention, the B portion can include a polycationic domain that is positively-charged at physiological pH. The B portion can include at least one domain that enables translocation of the B-Ab portion across a cell membrane. In addition to the translocation domain, the B portion can also include a domain that enables translocation of the B-Ab portion across the nuclear membrane. The B portion can include one or more basic or positively-charged amino acids, such as arginine, histidine and lysine. The B portion can include other amino acids, however, that are not basic. Additionally, the B portion can include other moieties, such as positively-charged moieties. In one example of the present invention, the amount of negative charge in the A portion can be approximately the same as the amount of positive charge in the B portion.

B portion can be linked to both the X portion and the Ab portion by covalent linkages. In aspect of the application, the B portion can be linked to the Ab portion using maleimide-thiol conjugation. By way of example, epsilon maleimido capropic acid (EMCA) can be attached to the B portion, and a thiol group can be introduced onto the Ab portion. The EMCA attached to the B portion can then be covalently bonded to thiol of the Ab portion.

In another aspect, the A portion can include a polyanionic domain that is negatively-charged at physiological pH. The A portion can be linked (e.g., covalently) to the X portion and can include one or more acidic or negatively-charged amino acids, such as glutamate or aspartate. The A portion can include other amino acids, however, that are not acidic. Additionally, the A portion can include other moieties, such as negatively-charged moieties. Including the acidic A portion as part of the ACPP can effectively inhibits or prevents the uptake of the B-Ab portion into cells. Such a block that would otherwise be affected by the basic B portion may be termed a “veto” of the uptake by the acidic A portion.

The A and B portions can include either L-amino acids or D-amino acids. In one example of the present invention, the A and B portions can comprise D-amino acids to minimize immunogenicity and non-specific cleavage. D-amino acids may be used to form all or only a portion of the A and B portions as cellular uptake of oligo-D-arginine sequences is known to be as good as (or better than) that of oligo-L-arginines. A composition having the generic structure A-X—B-Ab can be effective to deliver the Ab portion into a cell where the A portion is at the N-terminus or the C-terminus, i.e., either orientation of the peptide bonds is permissible. Where the X portion is a peptide cleavable by a protease (described below), however, the C-terminus of the X portion may be joined to the N-terminus of the B portion so that the new N-terminus created by cleavage of the X portion can contribute an additional positive charge to the positive charges already present in the B portion.

In another aspect, the X portion can be designed for cleavage in the presence of particular conditions or in a particular environment, such as the extracellular environment. For example, the X portion may comprise a plurality of amino acids that are cleavable by proteases or other enzymes found on the surface of neoplastic cells or released near neoplastic cells. In this case, the cleveable linker can include an amino acid sequence that is recognized and cleaved by a protease so that proteolytic action of the protease cleaves the X portion. The location of the X portion can be varied so that cleavage of the X portion allows separation of the A portion from the B-Ab portion. When separated from the A portion, the normal ability of the B portion to affect the uptake of the Ab portion into cells is regained. Such cellular uptake can occur near the location of the cleavage event. Thus, the amino acid sequence of the X portion can be such that the X portion is cleaved at or near a target cell (e.g., a neoplastic cell) to effectively direct uptake of the B-Ab portion into the target cell.

The capacity of solid tumors to invade surrounding tissue and to metastasize is correlated with the formation and degradation of structural elements in the vicinity of the tumor cells. One important class of signals is the hydrolytic activity of proteases, which are very important in the invasive migration of metastatic tumor cells. Four different classes of proteases are involved in tumor cell invasion and metastasis: (1) serine proteases; (2) metalloproteases (MMPs); (3) cysteine proteases; and (4) aspartyl proteases.

In one example, proteases (e.g., plasmin, trypsin, neutrophil elastase) may cleave the X portion to separate the A portion from the B-Ab portion and thereby allow cellular uptake of the B-Ab portion. Such uptake of the B-Ab portion is typically in the vicinity of the proteases that cleave the X portion. Thus, the ACPP of the present invention is able to direct cellular uptake of the B-Ab portion to neoplastic cells having active proteases in their extracellular environment.

For example, an X portion comprising the amino acid sequence RLQLKL (SEQ ID NO: 1) may be cleaved by serine proteases as described by Whitney, M. et al., “Parallel in vivo and in vitro selection using phage display identifies protease dependent tumor targeting peptides” J Biol Chem., In Press (published on-line May 11, 2010) (hereinafter, “Whitney et al.”). As disclosed by Whitney et al., an ACPP containing SEQ ID NO: 1 as the X portion was not cleaved by MMPs or various coagulation factors, but was efficiently cleaved by plasmin and elastases, both of which have been shown to be aberrantly over-expressed in tumors. Thus, an ACPP that includes an X portion having the sequence of SEQ ID NO: 1 can selectively deliver the B-Ab portion to neoplastic cells that overexpress proteases (e.g., serine proteases) in vivo.

In another example, at least one standard amino acid of SEQ ID NO: 1 can be replaced with a non-standard amino acid to further enhance enzyme selectivity and specific tumor uptake of an ACPP. For instance, the X portion can include a non-standard amino acid, such as N^{ε}-acetyl-lysine (SEQ ID NO: 2). As disclosed by Whitney et al., a ACPP including SEQ ID NO: 2 as the X portion was efficiently cleaved by tumor extracts and elastases but had reduced cleavage by liver and kidney extracts. Since it is known that liver cells can produce high levels of proteases, an ACPP that includes an X portion having SEQ ID NO: 2 can mitigate or prevent delivery of a B-Ab portion into non-neoplastic cells (e.g., liver and/or kidney cells).

In another example, MMPs may be used to cleave the X portion and separate the A portion from the B-Ab portion. This may allow cellular uptake of the B-Ab portion as a result of the enzymatic action of the MMPs. Since uptake is typically in the vicinity of the MMPs that can cleave the X portion, the ACPP of the present invention can direct cellular uptake of the B-Ab portion to specific cells, tissues, or regions having active MMPs in the extracellular environment. Examples an amino acid sequences that can comprise the X portion, and which MMPs can cleave, are disclosed in U.S. Pat. No. 7,431,915 to Jiang et al. (hereinafter, “the '915 patent”), the entirety of which is hereby incorporated by reference.

It will be appreciated that ACPPs described herein may be synthesized by standard synthetic techniques, such as solid phase synthesis (e.g., solid phase peptide synthesis). Examples of solid phase synthesis schemes are disclosed in the '915 patent, as well as by Whitney et al.

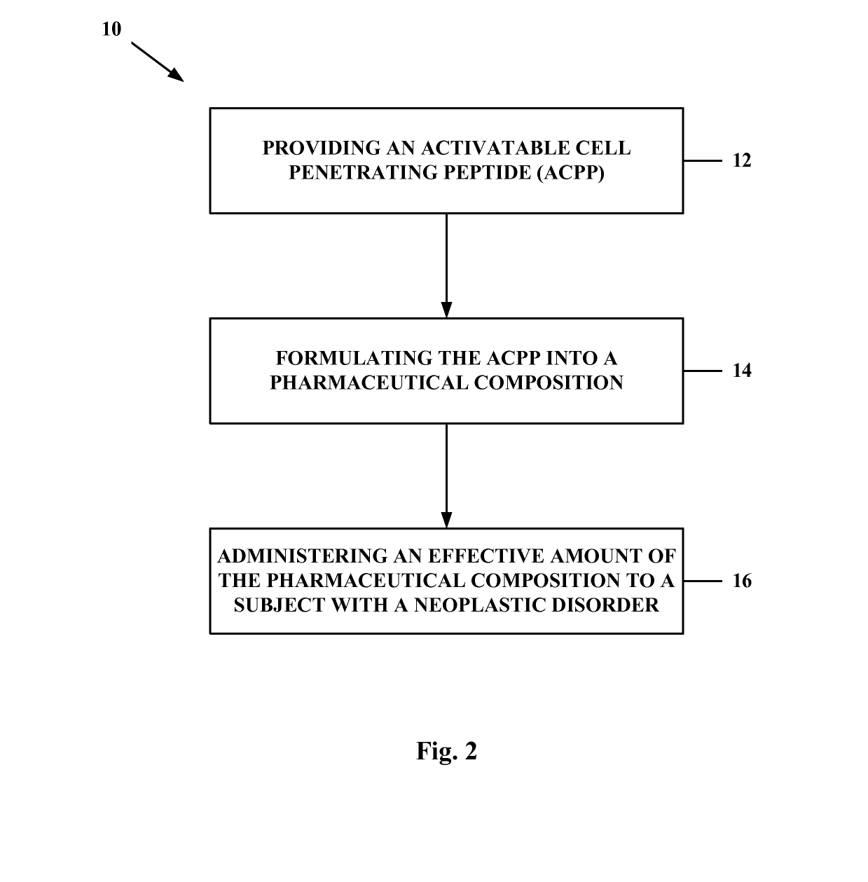

FIG. 2 is a flow diagram illustrating a method 10 in accordance with another aspect of the application for treating a subject with a neoplastic disorder. Generally, pathologies, including neoplastic disorders, treatable by the method 10 can include disease states in which there are cells and/or tissues that proliferate abnormally. One example of a neoplastic disorder is a tumor. The tumor can include a solid tumor, such as a solid carcinoma, sarcoma or lymphoma, and/or an aggregate of neoplastic cells. The tumor may be malignant or benign, and can include both cancerous and pre-cancerous cells. In one example of the present invention, the neoplastic disease can include breast cancer.

At step 12, the method 10 can include providing a composition for treating neoplastic disorders. The composition can have the general structure as shown in FIG. 1 and described above. For example, the A portion can comprise at least one acid amino acid residue, the B portion can comprise at least one basic amino acid residue, and the X portion can include a plurality of amino acids that are cleavable by a gene expression product of at least one neoplastic cell. The Ab portion of the composition can be selected based on the particular neoplastic disorder to be treated. To treat breast cancer, for example, the Ab portion can comprise the antibody aSSB70C. The antibody aSSB70C binds to RPA70, which is an essential gene product over-expressed in BRAC1^{mut} tumors (Basilion, J. P. et al., Mol Pharma. 56(2):359-369, 1999). Additionally, the antibody aSSB70C has been shown to have a neutralizing activity against RPA70 by inhibiting replication of SV40 DNA (Kenny, M. K. et al., J Biol Chem. 265(13):7693-7700, 1990).

At Step 14, the composition can be formulated into a pharmaceutical or therapeutic composition by compounding the composition with at least one pharmaceutically acceptable carrier. Pharmaceutically acceptable carriers are known in the art and may include any material or materials which are not biologically or otherwise undesirable, i.e., the material may be incorporated or added into the composition without causing any undesirable biological effects or interacting in a deleterious manner with any of the other components (i.e., the nanobubbles) of the composition. When the term “pharmaceutically acceptable” is used to refer to a pharmaceutical carrier, it can be implied that the carrier has met the required standards of toxicological and manufacturing testing or that it is included on the Inactive Ingredient Guide prepared by the U.S. Food and Drug administration.

After formulating the composition of the present invention into a pharmaceutical composition, an effective amount of the composition can be administered to the subject. The location(s) where the composition is administered may be determined based on the subject's individual need, such as the location of a tumor (e.g., the position of a tumor, the size of a tumor, and/or the location of the tumor on or near a particular organ). For example, the composition may be injected directly into a tumor (i.e., intratumorally). Alternatively, the composition may be injected intravenously into the subject. It will be appreciated that other routes of injection may be used including, for example, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal routes.

Upon administration to the subject, the intact ACPP couples to the antibody or fragment thereof would not be able to enter mammalian or non-neoplastic cells because of the presence of the A portion and the lack of extracellular enzymes capable of cleaving the X portion. However, when the ACPP is near or in the extracellular environment of a neoplastic cell or cells, cleavage of the X portion would occur as a result of certain overexpressed gene products (e.g., proteases). Consequently, cleavage of the X portion allows translocation of the B-Ab portion into the neoplastic cell (or cells) to affect targeted intracellular delivery of the Ab portion.

In one example, an effective amount composition comprising an ACPP can be intravenously administered to a subject with breast cancer. The A portion of the ACPP can comprise a polyanionic domain, the X portion can comprise the sequence of SEQ ID NO: 1, the B portion can comprise a polycationic domain, and the Ab portion can comprise the antibody aSSB70C. Upon administration to the subject, the intact ACPP would not be able to enter mammalian or non-breast cancer cells because of the presence of the A portion and the lack of extracellular enzymes (i.e., proteases) capable of cleaving the X portion. However, since breast cancer cells are known to overexpress proteases, ACPPs in the extracellular environment of the breast cancer cells would have their X portion cleaved by the overexpressed, extracellular proteases. Cleavage of the X portion would remove the A portion and enable the B-Ab portion to translocate into the breast cancer cells. Once the B-Ab portion has translocated into a breast cancer cell, the antibody aSSB70C can bind to and neutralize its target, i.e., RPA70. Neutralization of intracellular RPA70 can inhibit replication of SV40 DNA and thereby prevent or mitigate further replication of the breast cancer cells.

From the above description of the invention, those skilled in the art will perceive improvements, changes and modifications. For example, it will be appreciated that other arrangements of the A, B, X, and Ab portions beyond the one shown in FIG. 1 are possible, such as those disclosed in the '915 patent. Such improvements, changes, and modifications are within the skill of those in the art and are intended to be covered by the appended claims.

### Example

### Targeting of Essential Gene Products Cell Penetrating Neutralizing Antibodies

Abbreviations: Standard one letter or three letter codes for amino acids. Lower case one letter amino acids denotes D-isomer of amino acids. Unnatural residues are as indicated: Aca, amino caproic acid. EMCA, 6-maleimidocapropic acid; CPP, cell penetrating peptide.

### Design and Synthesis of Targeted Cell Penetrating Antibody

Delivery of antibody across the cell membrane had been achieved by employing various CPPs (e.g., TAT, penetratin, and polyarginine). However, these cell penetrating antibody bioconjugates do not discriminate to target tumor cells from normal cells. To introduce a selectivity component, the active cationic moiety of the CPP is neutralized with an anionic sequence. In addition, the cationic and anionic sequences are linked by a protease sensitive sequence. Upon proteolytic processing, the cationic sequence is activated for intracellular delivery of the antibody. To target tumor cells, the protease sensitive linker sequence is based on MMP-2 substrate. MMP-2 is a protease enzyme that is abundant in the around the extracellular tumors and has been previously used to activate inactive agents. Here we chose nona-D-arginine as the polycationic peptide and nona-D-glutamic acid as the polyanionic peptide. D-isomers of arginine and glutamic acid were employed for stability from proteolytic enzymes. MMP-2 cleavable substrate sequence is PLGLAG. Aca group is used as spacer linker group. Due to the presence of carboxylates from the polyglutamic acid, EMCA was employed as the key thiol functional group for selective covalent bond with antibody. To monitor conjugation of peptide to antibody and subsequent analyses of cell penetration and localization an additional lysyl residue and added for labeling with fluorophores (e.g., Cy 5). Compound 1 consisted of polyarginine, Cy5 label, and maleido group. Compound 2 consisted of an MMP-2 cleavable sequence flanked by polyarginine and polyglutamic acid sequences, Cy5 label, and a maleido group. Compound 3 is an analogue of Compound 2 with a quenching dye Qsy-21.

### Peptide Synthesis

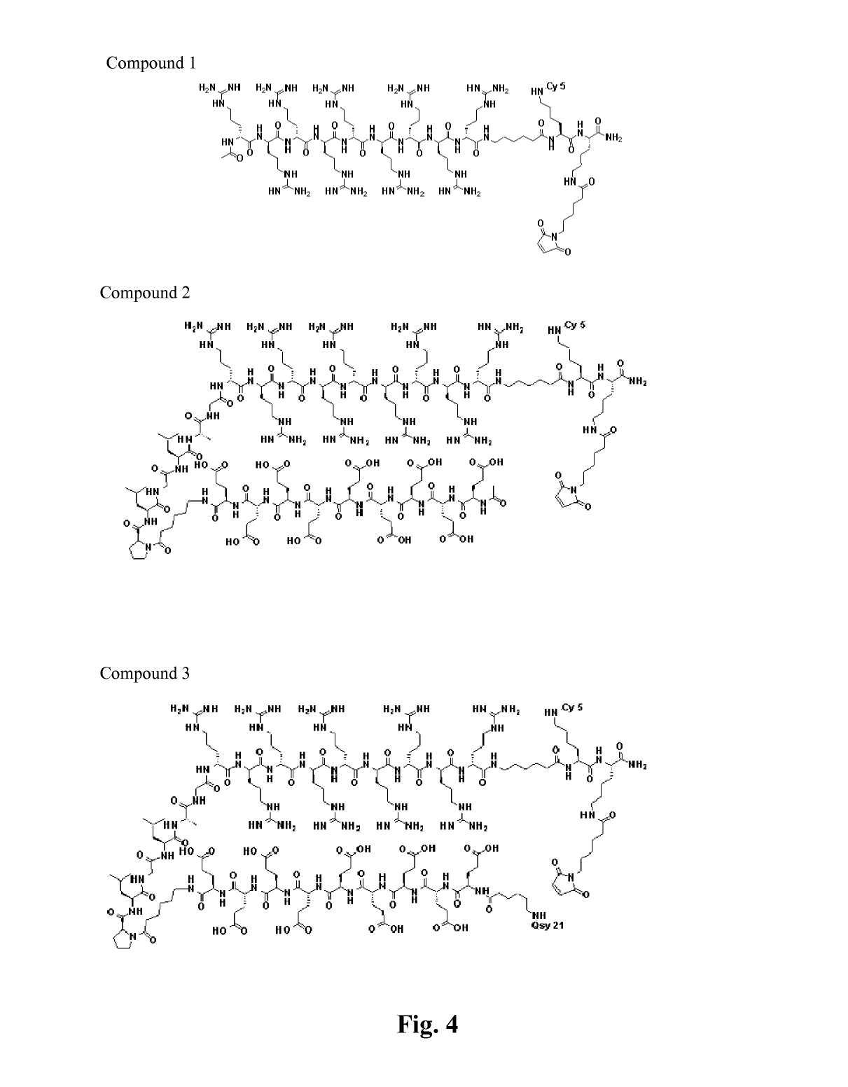

All peptides were synthesized manually using Fmoc protected amino acids on Rink-amide CLEAR resin. Peptide synthesis reagents were purchased from Peptides International (Louisville, Ky.). Standard Fmoc solid phase synthesis protocols were used. Fmoc-D-Arg(Pbf)-OH and Fmoc-Aca-OH were purchased from Peptides International. Fmoc-Lys(N^{epsilon}-Mtt)-OH and Fmoc-Lys(N^{epsilon}-Alloc)-OH_were purchased from Novabiochem. The linear peptide was synthesized on resin and acetylated at the N-terminal end with acetic anhydride. The orthogonal Alloc deprotection was achieved by Pd⁰ on resin and the resulting free amine was coupled to EMCA-NHS ester. Global deprotection and cleavage from resin was achieved with 95% trifluoroacetic and water and triisopropylsilane as scavenger. The peptide was purified by reverse-phase HPLC. The free amino peptide was selectively conjugated to Cy5-NHS ester (GE Amersham) (FIG. 4). The final fluorescently labeled peptides were purified by HPLC and characterized by MALDI mass spectrometry: Compound 1, Ac-rrrrrrrrr-Aca-K(N^{epsilon}-Cy5)-K(N^{epsilon}EMCA)-amide (MW=2666.53); Compound 2, Ac-ddddddddd-Aca-PLGLAG-rrrrrrrr-Aca-K(N^{epsilon}-Cy5)-K(N^{epsilon}EMCA)-amide (MW=4446.3); Compound 3, Qsy21-Aca-ddddddddd-Aca-PLGLAG-rrrrrrrr-Aca-K(N^{epsilon}-Cy5)-K(N^{epsilon}Ac)-amide (MW=5032.8)

### Bioconjugation to Antibody

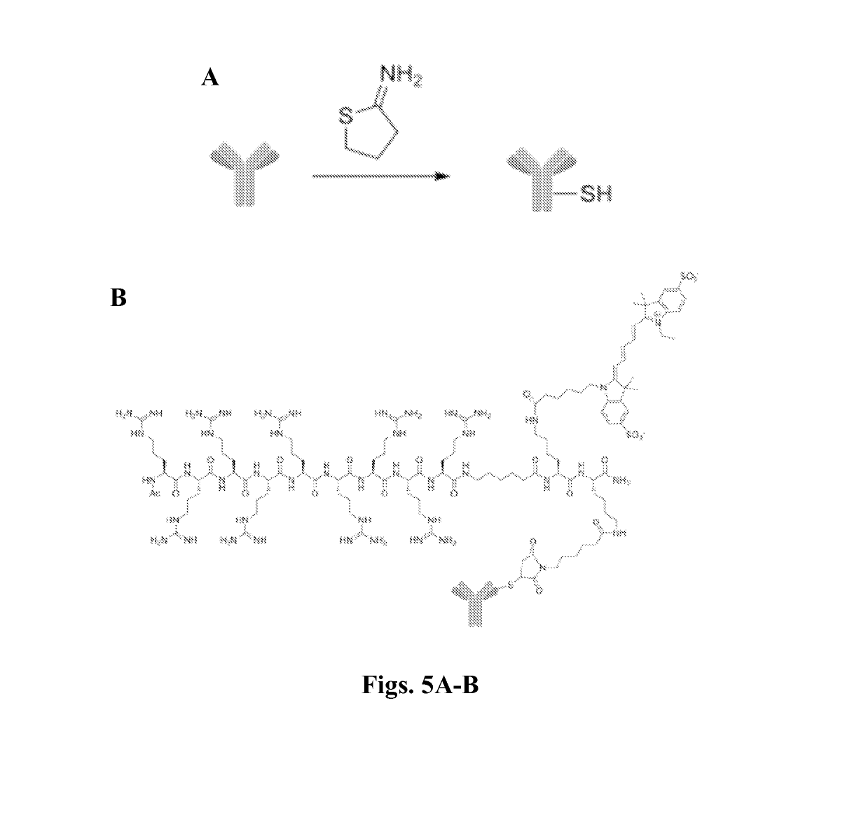

Pure immunoglobulin antibodies (mouse and donkey IgG, anti-His, and anti-RPA70 clone 2H10) were purchased from commercial sources. For the bioconjugation, the antibodies was maintained in degassed PBS (pH 8) and was reacted with thiolane (Sigma) 20 equivalents for 1 hour at room temperature. Excess unreacted thiolane was removed by size exclusion centrifugation through PD-10 spin column (GE Healthcare). The thiolated antibodies was incubated with 5-10 fold excess of compound 1 at room temperature for 1 hour to form a covalent bond between antibody thiol and the maleimido group of compound 1 (FIG. 5). The resulting cell penetrating antibodies bioconjugate was purified with PD-10 spin columns (GE Healthcare) and further concentrated on YM-30 Microcon (MWCO 30K (Amicon)). Dye to antibody ratio was estimated by determining the concentration ratio of the fluorophore (e.g., ext. coeff. 250K for Cy5 at A_{650 nm}) for 1 to the concentration of the antibody (ext. coeff 170K at A_{280 nm}). Labeling ranges from 0.2-13 peptide per antibody.

### Cell Penetrating Antibodies In Vitro Assays

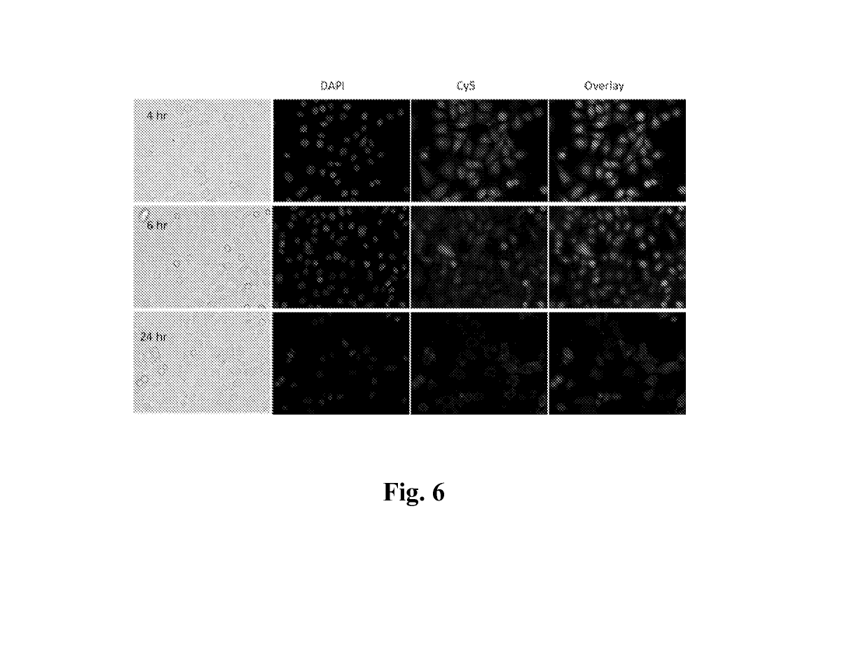

Our first step in these studies was to examine if a cell penetrating peptide that we had synthesized would be able to enter into cells. Compound 1 was incubated with MDA-MB-468 cells. FIG. 6 shows that at 4 hours the peptides can already be observed inside the cell. However, after 24 hours, the fluorescence signal has significantly decreased. This indicates that we should consider observing penetration of peptide/antibody conjugates at earlier time points, e.g., 4 or 6 hours. The overlay on the third column suggests nuclear penetration of the peptides; however confocal microscopy should be done to confirm this.

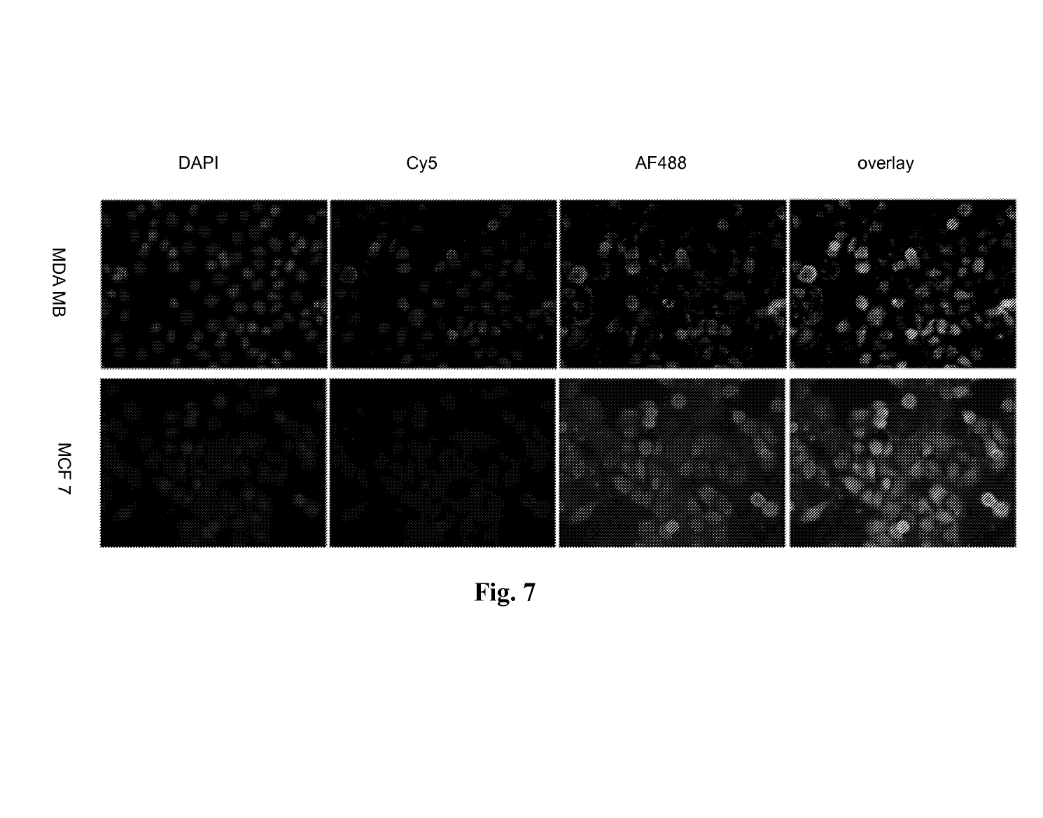

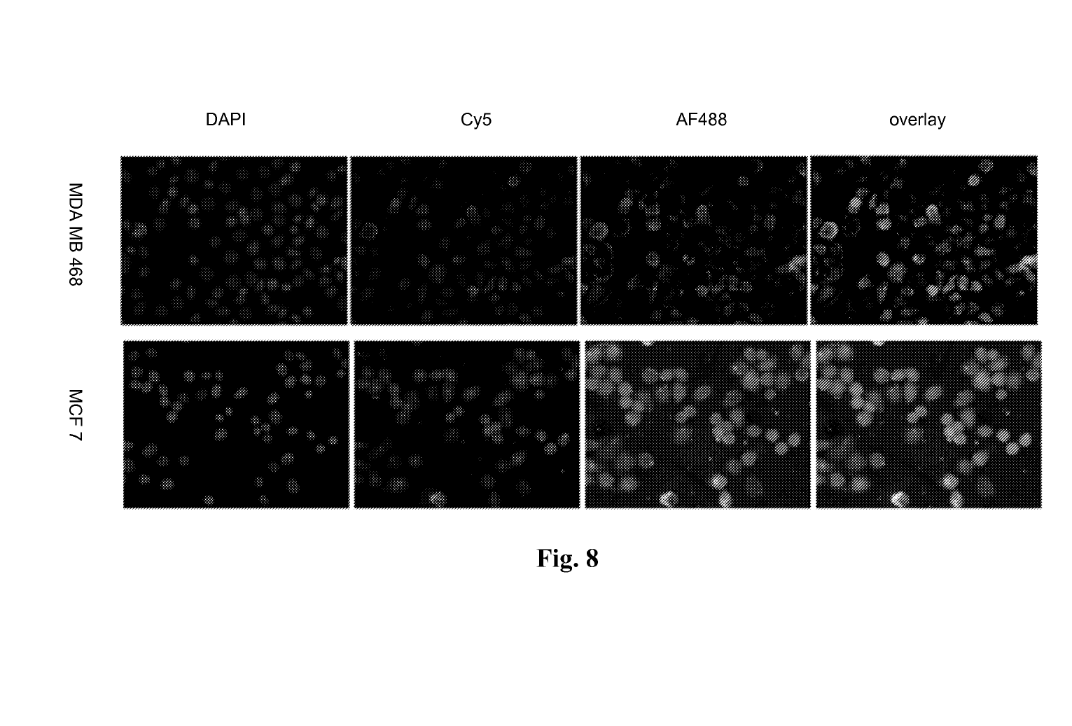

Our next step in these studies was to determine if the cell penetrating peptide when conjugated to antibodies would drive the complex into cells. For this study we conjugated either anti-RPA1 antibody or anti-His antibody (control) to compound 1 and applied them to cells. FIGS. 7 and 8 shows the cell penetration of bioconjugated anti-RPA1 and anti-His antibody in MDA-MB-468 and MCF-7 cells after 4 hours of incubation. To confirm that the antibody cell penetrating peptide bioconjugate is in the cytosol, the mouse IgG was counterstained with an anti-mouse AlexaFluor 488 label. These qualitative microscopic images suggest that the cell penetrating antibody bioconjugates are able to penetrate the cell.

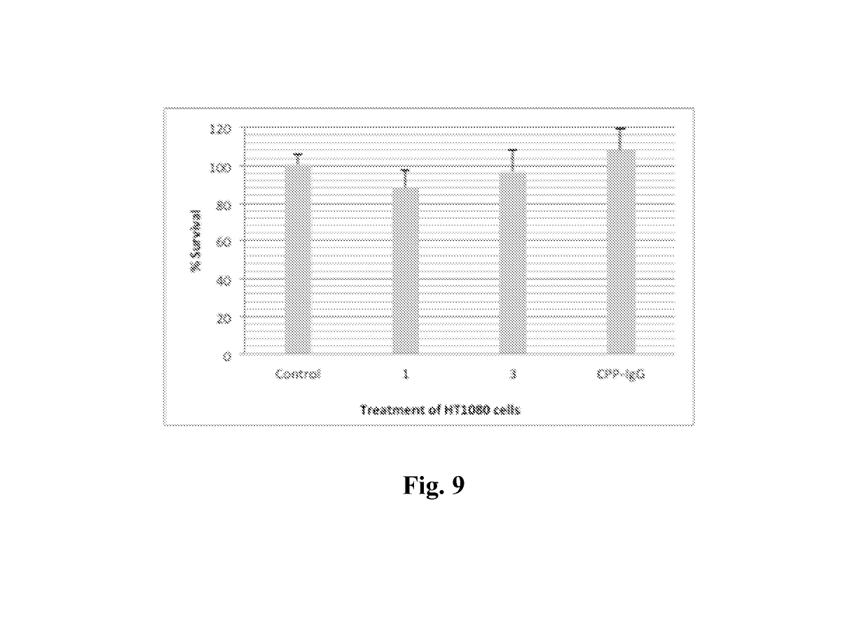

We also attempted to determine if compound 3, which should not cross the membrane of cells that do not express MMP2, could enter cells. HT1080 cells, which express MMP2, were incubated with compounds 1, 3, or compound 1 conjugated to non-specific mouse immunoglobulin antibody. In the case of unconjugated as well as conjugated compound 1, the Cy labeled peptide entered into cells. Compound 3 also entered into cells, but to a lessor extent, however, this was significantly greater than background fluorescence. This is most likely due to incomplete activation of the cloaked CCP. We also determined whether the peptides and the cell penetrating antibodies were toxic to these cells. Compounds 1, 3, or compound 1 conjugated to non-specific mouse immunoglobulin antibody were incubated with HT1080 cells (FIG. 9). None of the incubations resulted in toxicity to the cells, and all remained within the rage of the control cells.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention, the following is claimed:

1. A composition for treating a neoplastic disorder comprising a structure A-X—B-Ab, wherein:
Ab portion is a neutralizing antibody and/or fragment thereof that binds to an essential and/or neoplastic disorder-specific intracellular target;
B portion is a polycationic domain comprising at least one basic amino acid residue, which is effective to transport the Ab portion across a membrane of at least one mammalian cell;
A portion is a polyanionic domain comprising at least one acidic amino acid residue, which when linked with the B portion is effective to inhibit or prevent uptake of the B-Ab portions by the at least one mammalian cell; and
X portion is a cleavable linker comprising a plurality of amino acid residues, which is cleavable by a gene expression product of at least one neoplastic cell.

2. The composition of claim 1, the gene expression product of the at least one neoplastic cell comprising a protease.

3. The composition of claim 1, the X portion comprising the sequence of SEQ ID NO: 1 (RLQLKL).

4. The composition of claim 1, the X portion comprising the sequence of SEQ ID NO: 2 (RLQLK(Ac)L).

5. The composition of claim 1, the essential and/or neoplastic disorder-specific intracellular target including a gene and/or gene product that is needed for cell survival.

6. The composition of claim 5, the essential and/or neoplastic disorder-specific intracellular target including replication protein A1, 70 kDa (RPA70).

7. The composition of claim 1, the B portion being linked to the Ab portion using maleimide-thiol conjugation.

8. A method for treating a neoplastic disorder in a subject, the method comprising:
administering to at least one neoplastic cell of the subject an effective amount of a composition having the structure A-X—B-Ab, wherein:
Ab portion is a neutralizing antibody and/or fragment thereof;
B portion is a polycationic domain comprising at least one basic amino acid residue, which is effective to transport the Ab portion across a membrane of at least one mammalian cell;
A portion is a polyanionic domain comprising at least one acidic amino acid residue, which when linked with the B portion is effective to inhibit or prevent uptake of the B-Ab portions by the at least one mammalian cell; and
X portion is a cleavable linker comprising a plurality of amino acid residues, which is cleavable by a gene expression product of the at least one neoplastic cell;
wherein the Ab portion binds to an essential and/or neoplastic disorder-specific intracellular target and thereby prevents replication of the at least one neoplastic cell.

9. The method of claim 8, the gene expression product of the at least one neoplastic cell comprising a protease.

10. The method of claim 8, the X portion comprising the sequence of SEQ ID NO: 1 (RLQLKL).

11. The method of claim 8, the X portion comprising the sequence of SEQ ID NO: 2 (RLQLK(Ac)L).

12. The method of claim 8, the essential and/or neoplastic disorder-specific intracellular target including a gene and/or gene product that is needed for cell survival.

13. The method of claim 12, the essential and/or neoplastic disorder-specific intracellular target including RPA70.

14. The method of claim 8, the B portion being linked to the Ab portion using maleimide-thiol conjugation.

15. A composition for treating a neoplastic disorder comprising an activatable cell penetrating peptide coupled to antibody and/or fragment thereof to an essential gene and/or gene product of a neoplastic cell.

16. The composition of claim 15, the activatable cell penetrating peptide comprising a linker that is cleavable by a gene expression product of the at least one neoplastic cell

17. The composition of claim 16, the gene expression product of the at least one neoplastic cell comprising a protease.

18. The composition of claim 15, the essential gene and/or gene product is a gene and/or gene product needed for cell survival.

19. The composition of claim 15, gene and/or gene product comprising replication protein A1, 70 kDa (RPA70).

20. The composition of claim 15, the antibody being linked to the activatable cell penetration peptide using maleimide-thiol conjugation.


SEQUENCE LISTING (2 sequences)

SEQ ID NO: 1 - protein, 6 aa, Homo sapiens
RLQLKL

SEQ ID NO: 2 - protein, 6 aa, Homo sapiens
  MOD_RES (5)..(5): ACETYLATION
RLQLKL